(19) United States
(12) Patent Application Publication
(10) Pub. No.: US 2025/0237638 A1
(43) Pub. Date: Jul. 24, 2025

(54) METHOD AND SYSTEM FOR DETERMINING COLOR MATCH FOR SURFACE COATINGS

(71) Applicant: SWIMC LLC, Cleveland, OH (US)

(72) Inventors: Klaas DE HAAS, Warnsveld (NL); Asei AKANUMA, London (GB); Daniel STAMATE, Orpington (GB); Frederick DE GREEF, Amsterdam (NL)

(21) Appl. No.: 19/063,731

(22) Filed: Feb. 26, 2025

Related U.S. Application Data
(63) Continuation of application No. 18/704,618, filed on Jan. 1, 0001.
(60) Provisional application No. 63/271,462, filed on Oct. 25, 2021.
(60) Provisional application No. 63/291,056, filed on Dec. 17, 2021.
(60) Provisional application No. 63/309,062, filed on Feb. 11, 2022.
(60) Provisional application No. 63/329,976, filed on Apr. 12, 2022.

(51) Int. Cl. G01N 33/32; G01N 21/25; G06N 3/048

(52) U.S. Cl. CPC G01N 33/32; G01N 21/25; G06N 3/048; G01N 2201/1296

(57) ABSTRACT

One or more techniques and/or systems are disclosed for providing for improved color prediction from a known coating formulation by using Artificial Intelligence/Machine Learning models. A machine learning models can be used to improve color match accuracy between a given formula and the resulting application of the colored coating. Using this approach, a single color prediction model can be used for a set of multiple binders and a set of colorants to predict a more accurate color prediction, instead of separately training separate models for each different color coating. As an example, a single model can be trained with all the sets of target binders and all the sets of target colorants; and this model can be used to accurately predict the resulting color for a proposed formulation of a coating input to the single trained model.

### RELATED APPLICATION DATA

This application is a continuation of and claims priority to U.S. patent application Ser. No. 18/704,618, filed Apr. 25, 2024. Application Ser. No. 18/704,618 is a National Phase Application of International Application No. PCT/US2022/046394 filed Oct. 12, 2022, which claims priority to and benefit of U.S. Provisional Application 63/271,462 filed on Oct. 25, 2021, U.S. Provisional Application 63/291,056 filed on Dec. 17, 2021; U.S. Provisional Application 63/309,062 filed on Feb. 11, 2022; and U.S. Provisional Application 63/329,976 filed on Apr. 12, 2022. The entireties of the aforementioned applications are incorporated by reference herein.

### BACKGROUND

Surface coating materials, such as paint, are available for different types of applications, and can be provided in a variety of colors, hues, and tints, etc. In painting applications, for example, specialty paints can include a base paint mixed with colorants to a desired final color for a target project, resulting in a desired appearance on the target surface. As such, the final paint color and resulting appearance can be specific to the project and target application. Color matching to the desired resulting color for an application is often performed using a technician colorist's skills and trial-and-error, mixing and matching colorant and base to obtain the desired result. Further, computer assisted color matching can utilize traditional analytical models based on optical matching to come up with a possible match. The production of colorant to match to target coating application can be quite labor intensive.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems described herein can be utilized for improving color match accuracy and efficiency by using Artificial Intelligence/Machine Learning models. In some implementations, artificial intelligence/machine learning models can be used to improve color match accuracy between a given formula and the resulting application of the colored coating. This approach can use a single color prediction model for a set of multiple binders and a set of colorants to provide an accurate color prediction, and reduce time and resources needed to separately train separate models for each different color coating. As an example, a traditional model is assigned to, and has to be separately trained for, a single coating comprising a different combination of binder with a set of colorants. In some implementations, a single model can be trained with all the sets of target binders and all the sets of target colorants; and this model can be used to accurately predict the resulting color for a proposed formulation of a coating input to the single trained model.

In one implementation for a method or system for color matching to a coating formulation, a coating formulation can be input into a trained machine learning color prediction model. In this implementation, the coating formulation comprises a binder component selected from a set of binder components and a colorant component selected from a set of colorant components, resulting in a color match prediction for the coating formulation. Further, the trained machine learning color prediction model can be trained over the set of binder components that are used in a plurality of coating formulations. Additionally, the machine learning color prediction model can be trained over the set of colorant components that are used in a plurality of coating formulations. This can result in the trained machine learning color prediction model able to predict the color match for the plurality of coating formulations.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 illustrates various flow diagrams for predicting a color of a coating formulation.

FIG. 2 illustrates various flow diagrams for training a color predicting machine learning model.

FIGS. 3A, 3B, and 3C illustrate graphical representations of results identified from various color prediction models.

FIG. 4 is a flow diagram illustrating an example method for predicting a color of a given coating formulation.

FIG. 5 illustrates a schematic diagram for training a color predicting machine learning model, based on one or more techniques described herein.

FIG. 6 is a block diagram of an example system suitable for implementing various examples color prediction described herein.

FIG. 7 is a block diagram of an example computing environment suitable for implementing various examples of well-being monitoring.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

Color matching is a specialized task by which a colorist mixes a set of constituents having color properties (e.g., pigments, etc.) to form colorants (e.g., using a colorant recipe), which can be mixed into a coating formulation in different proportions until a suitable visual match is achieved between the mix and a desired or target color. That is, for example, a colorant recipe may provide the color for a coating formulation (e.g., comprising the colorant and other coating components). The resulting coating product comprises a formula or formulation, sometimes called a mixture of a colorant recipe into coating formulation, which can comprise a number of mixing ratios of the colorants (e.g., or pigments) used to obtain the color match. Color matching is therefore a considerably laborious task and is achieved through a combination of the colorist's skills and trial-and-error. To assist the colorists, and to speed up the process of discovering formulations, computer colorant recipes are often used that utilize various color prediction software. This type of software may allow for the prediction of resulting color given a formulation, which are often based on analytical models. As an example, a relatively common method of computer colorant development is to implement an analytical model based on the radiation transfer theory known as Kubelka-Munk (K-M). K-M based models allow for prediction of the spectral reflectance of a mixture of colorants by characterizing them according to two coefficients, absorption (K) and scattering(S). Though the K-M theory is favored for its simplicity and ease of use, in many cases the approximations alone are not sufficiently accurate for real world applications.

One disadvantage of existing machine learning modeling is that a large amount of data is typically needed to provide accurate and desired results, at least results that equal or improve upon non-machine learning modeling. Further, with regard to colored surface coatings, each coating contains one or more binders and one or more colorants. Existing models for color prediction of a colored coating need to be trained for each different set of binders, over a set of colorants. This leads to training of several different models to achieve desired color predictive matching. To overcome these issues, a hybrid approach may be used that allows for a reduced or limited data set for training to merely a single model, while providing improved accuracy over traditional, non-machine learning prediction.

As an example, output data from a traditional, non-machine learning technique (e.g., K-M based model) can be used as initial input to a trained machine model, resulting in a color matching prediction that corrects a prediction made by the traditional model, whose accuracy is at least on par with the traditional, stand-alone machine learning models. By using the initial input from a traditional model, the trained model may not need as much training data to reach an improved (e.g., corrected) prediction. Further, as more data is provided to the machine learning model, prediction performance will improve. As such, machine learning models for color matching prediction can be utilized for coatings that may have limited available data (e.g., either new products or products that simply have a limited number of colors).

Further, in one aspect, colored coating disposed on a surface can be measured using a variety of devices, as well as using the human eye. As an example, based on human visual perception, color measurement can rely on a system of three color values which are referred to as tristimulus values (X, Y, Z). This measurement system attempts to represent or standardize the color receptors (cones) in the human eye, for example, as the “standardized observer” has a wavelength dependent sensitivity that deviates slightly from the human eye. Tristimulus values are a measure for light intensity based on the three primary color values (RGB), typically represented by X, Y, and Z coordinates. As one example, a spectrophotometer can simulate human eye technology to create accurate yet objective and quantifiable color measurement values. These measured values can be repeated for color quality and consistency in product development. Measurements can, among other things, consist of the visual part of the reflection spectrum—or sets of reflection spectra in case of effect pigments like metallic flakes—of the coating. Using the standardized observer and standardized light sources, this reflection spectrum can be translated into light source dependent tristimulus values (XYZ). This color space is based on human visual perception, where XYZ are representative for the three different types of color receptors (cones) in the human eye. The tristimulus values are used in color language, also referred to as the CIE color system and its associates CIE Lab color space, and is used to calculate and communicate color values, particularly when describing the color of a coating on a surface application. It should also be noted that trained colorists may be able to perform color correction merely using their own eyes.

The methods and systems disclosed herein, for example, may be suitable for use in color matching coatings (e.g., paints, stains, varnishes, chemicals, etc.) for different coating applications. For example, predictive colorant matching, such as a surface coating application, can utilize one or more input variables to generate a more accurate prediction of a color match for the desired use of the coating. It should be appreciated that the herein described examples can be used in different settings or environments for different types of coating applications and with different coating materials. The examples given herein are merely for illustration.

A method may be devised for accurately predicting a color match for a surface coating. An initial color match prediction can be provided using a traditional color matching technique, such as a human visual match and/or a computer modeling match (e.g., K-M based model using radiation transfer theory). The initial color match prediction (traditional model prediction) can be input to a trained color match model (e.g., trained using machine learning over a limited data set) to provide an adjusted (e.g., or corrected) color match.

That is, for example, ingredients for a color formulation (ingredients of a formulation), which may be used for a surface coating or colorant to mix with a coating base, can be provided to a traditional prediction model. The result of the traditional prediction model is an initial or first color prediction. Further, a color prediction machine learning model can be trained over a training set of data (e.g., of known formulations and results). As one example, the training set of data may be a limited or smaller set than is typically used to train a predictive machine learning model. In this example, the initial color prediction can be input to the trained machine learning model, along with the color formulation, and the trained machine learning model can provide an adjusted (e.g., corrected) or second color prediction.

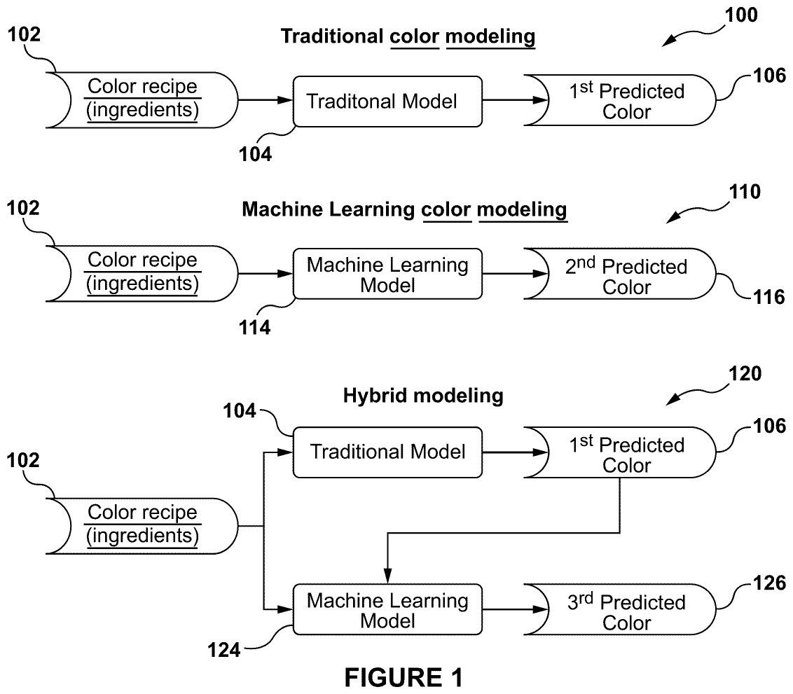

As one example, as illustrated in FIG. 1, using traditional color modeling 100, a color formulation 102 (e.g., ingredients of surface coating) is used as input to a traditional model 104, such as a human-based visual trial, and/or a computer-based method (e.g., K-M based model using radiation transfer theory). In this example, the output of the traditional model 104 is a first predicted color 106 for the color formulation 102. Further, using machine learning color modeling 110, the color formulation 102 is used as input to a first trained machine learning color model 114, trained to identify or predict a color for coating formulations. In this example, the output of the first machine learning color model 114 is a second predicted color 116 for the color formulation 102. Additionally or alternatively, using a new hybrid model, as further described herein, the color formulation 102 is used as input to the traditional model 104, and used as input to a second trained machine learning color model 124. In some implementations, the second trained machine learning model 124 may be the same as, or similar to, the first trained machine learning color model 114; and in other implementations the second trained machine learning color model 124 may be trained using different training data, and/or a smaller training set of data than the first trained machine learning color model 114. Typically, for example, the input to the second trained machine learning model 124 may comprise the color recolor recipe plus the first predicted color, thereby differing from the first trained machine learning color model 114. As illustrated in this example, the first predicted color 106 is also used an input to the second trained machine learning color model 124. The output of the second trained machine learning color model 124 is a third predicted color 126. In this way, in this example, the first predicted color 106 can be used as a basis for the second trained machine learning color model 124 selecting the third predicted color 126, based on the formulation 102. Essentially, for example, the second trained machine learning color model 124 can correct or adjust the first predicted color 106.

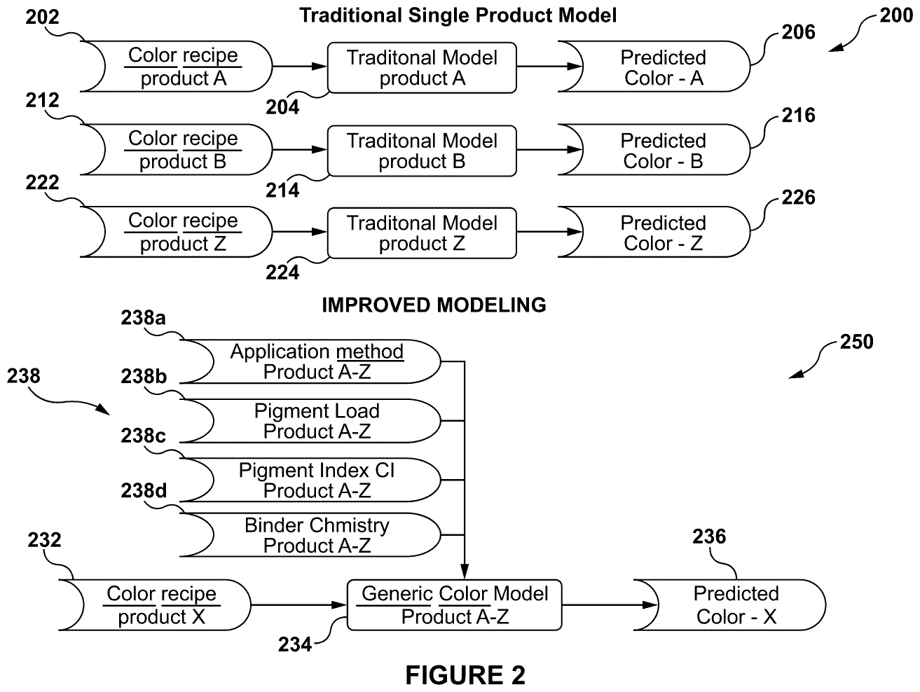

In one aspect, a trained machine learning color model can be trained to predict a color for myriad proposed color formulations, where the respective color formulations (e.g., for a surface coating) can comprise various pigments (or colorants) and pigment (or colorant) loads, various pigment indices, various binder chemistries, and may be used for various applications. As illustrated in FIG. 2, existing traditional single product models 200 (e.g., K-M based model using radiation transfer theory), can be used for predicting colors for a specific product (e.g., comprising a specific binder chemistry, for a target application). As illustrated, traditional color model A 204 can be used to predict a color for Product A formulations that all have binder chemistry A, but may have different pigment levels and indices. In this example, a color formulation for product A 202 can be input to the traditional color model for product A 204, resulting in predicted color for product A 206. Further, a traditional color model B 214 can merely be used for product B, inputting product B formulations 212, resulting in predicted colors for product B 216. Likewise, a traditional color model Z 224 can merely be used for product Z inputting product Z formulations 222, resulting in predicted colors for product Z 226. That is, existing traditional color models are useful for the ingredients that may go into their product, in order to predict a color for a particular formulation. However, in some implementations, this traditional single product modelling can be extended to Machine Learning, for example, by using an AI model instead of a traditional model like K-M based model using radiation transfer theory.

In one implementation of an improved trained machine learning color model 250, the myriad ingredient inputs 238 can be used to train the machine learning color model 234 that can be used for all of the products subjected to color prediction. That is, for example, the inputs 238 used to train the machine learning color model 234 can include the application methods 238a used for each of the products (A-Z) that will utilize the model 234; the expected pigment loads 238b that can be used for each of the products (A-Z) that will utilize the model 234; the expected pigment indices 238c that can be used for each of the products (A-Z) that will utilize the model 234; and the expected binder chemistries 238d that can be used for each of the products (A-Z) that will utilize the model 234. In this way, for example, a color formulation for any of the products (A-Z), such as a formulation for product X 232, can be input to the trained machine learning color model 234, resulting in a predicted color for product X 236. In some implementations, this type of trained machine learning color model 234 may be used for the machine learning color model (e.g., 124 of FIG. 1) in the hybrid color prediction described above and further herein. As described above, some machine learning color models may be trained on a limited data set based on the types of target products for the color prediction. In some implementations, properties for the target product can be used to train the color models may include, but are not limited to, gloss, refractive index, density, particle size, particle orientation distribution (effect colors), paint additives (e.g. dispersants that affect effective particle size), instruments (e.g., different type of measurements in database gloss included/excluded), pigment classification, supplier specific pigment treatment, production method (e.g., mill/beads-process settings), application method (e.g., spray/draw down), substrate including preparation (e.g. sanding of wood), layer build up (primer, clearcoat)

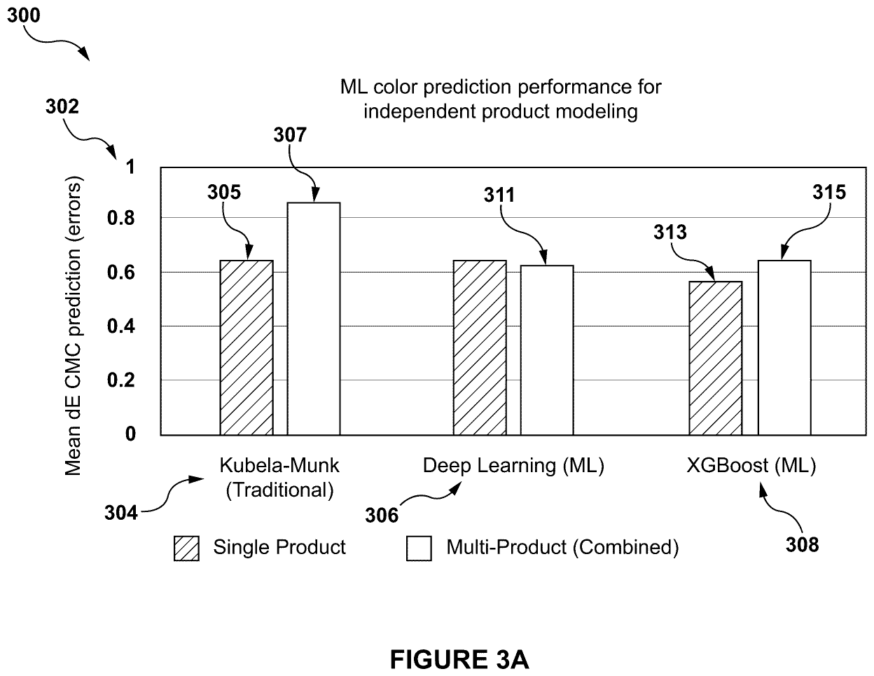

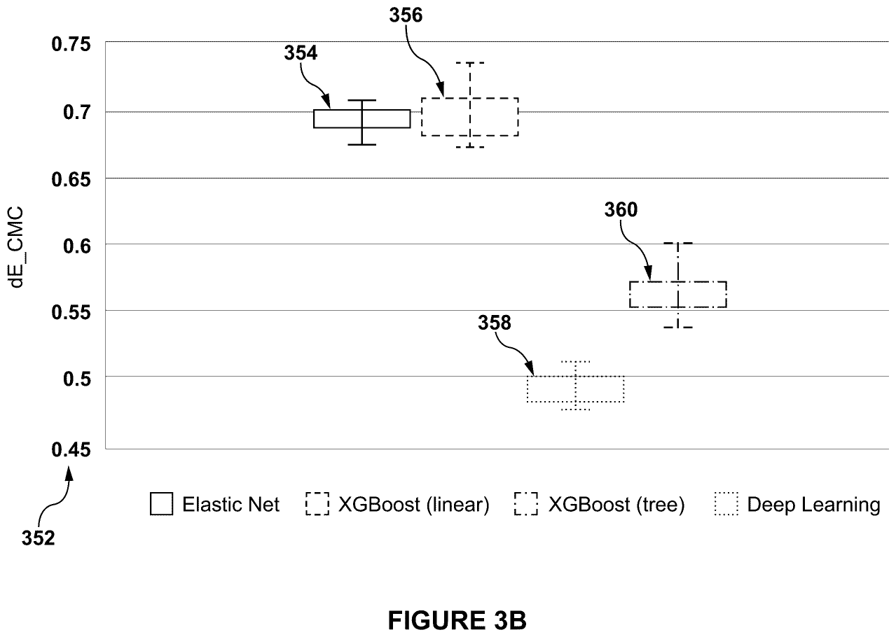

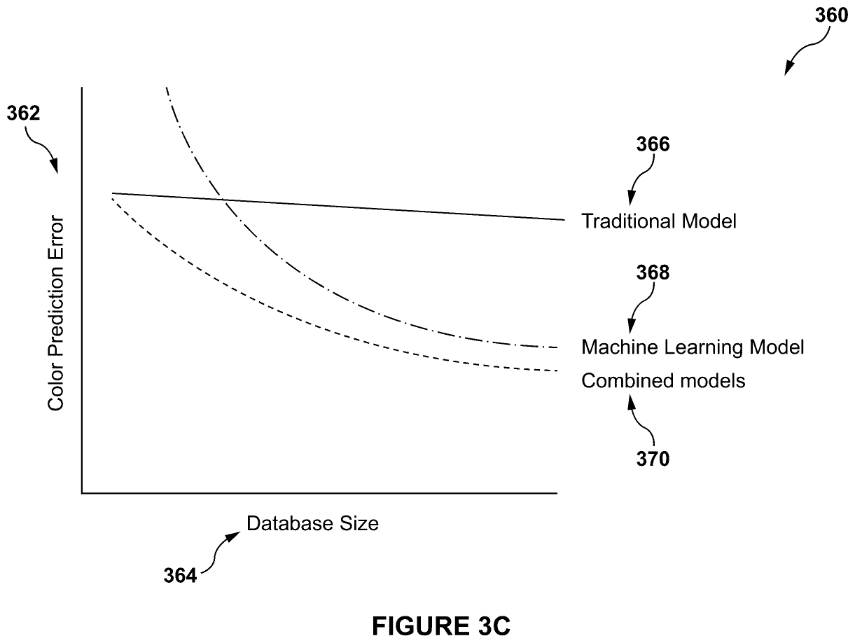

As illustrated in FIGS. 3A and 3B, the use of the trained machine learning color model (e.g., 124 of FIG. 1, 234 of FIG. 2) and the use of the hybrid color prediction method (e.g., 120 of FIG. 1) can result in a lower error rate than traditional color prediction models. The color prediction performance graph 300 in FIG. 3A shows an error rate 302 for various color prediction model's 304, 306, 308 performance, comparing independent product modeling 305, 309, 313, such as illustrated in 200 of FIG. 2 or 100, 110 of FIG. 1, to multi-product modeling 307, 311, 315, such as 250 of FIG. 2. In this example, the traditional color prediction model 304 (e.g., K-M model, such 104 of 100) indicates an error rate of about 0.65 mean dE_{CMC} for the traditional single product modeling 307 (e.g., 104 of FIG. 1, 204 of FIG. 2), and an error rate of about 0.9 mean dE_{CMC} for the traditional multi-product modeling 307 (e.g., using different products or product constituents, such as 238, in a generic model 234 of FIG. 2). For example, traditional single product modeling 307 implies that a traditional model like K-M based model using radiation transfer theory is used in model 234.

Further, a deep learning (DL) model 306 (a machine learning model described further below), indicates an error rate of less than 0.65 mean dE_{CMC} for the a single product modeling 309 (e.g., 114 of FIG. 1, 214 of FIG. 2), and an error rate that is similar for the multi-product modeling 311 (e.g., using combined, different product modeling, such as 250). That is, the multi-product trained DL model 311 (e.g., trained over various sets of the binders, pigment loads, pigment indices, and applications) has a lower error for predicting color for a variety of products than the traditional model 304 used to independently predict separate color for separate products 305. Additionally, an Extreme Gradient Boosting (XGBoost) model 308 (a machine learning model described further below), indicates an error rate of less than 0.60 mean dE_{CMC} for the a single product modeling 313, and an error rate of about 0.65 mean dE_{CMC} for the multi-product modeling 315 (e.g., using combined, different products). That is, the multi-product trained XGBoost model 315 (e.g., trained over various sets of the binders, pigment loads, pigment indices, and applications) has a lower error for predicting color for a variety of products than the traditional model 304 used to predict color for various products 307.

As an illustrative example, a coatings product line might have multiple products (e.g., for different applications) with a same set of colors available for the various products. In this example, the different products in the product line can comprise different binders (e.g., making up a set of binders), and may have the same set of colorants (e.g., making up the set of colorants). As illustrated by the performance graph 300, the result show that the performance of the multi-product machine learning models 311, 315 (e.g., trained over the set of binder and set of colorants for the product line) is at least as good or better than the traditional analytical model 304, single product performance 305, and with regard to deep learning model 306, the multi-product performance 311 is better than the sing product model 309 performance. However, as an example, as described herein, the deep learning multi-product model 311 is a single model that is trained over sets of multiple binders and sets of multiple colorants to achieve the improved prediction results. Where the single product models 305, 309 of the traditional analytical model 304 and deep learning model 306 comprise multiple models, one for each coating made of a different binder and colorant combination, which requires the training and application of multiple models instead of just the one provide by the deep learning multi-product model.

The color prediction performance graph 350 in FIG. 3B illustrates an error rate 352 for a hybrid color prediction model (e.g., 120 in FIG. 1), using various machine learning models. In this example, four different machine learning models were used in the hybrid modeling approach, including Elastic Net 354, XGBoost (linear model) 356, XGBoost (tree model) 358, and Deep Learning 360. As described in FIG. 1 above, using the hybrid model (e.g., 120) for color prediction, a first color prediction (e.g., 106), resulting from a traditional model (e.g., 104), is input to the machine learning color model (e.g., 124), along with the formulation (e.g., 102) for the colored product for which the prediction is desired. As illustrated, the Elastic Net 354 model used in the Hybrid Model approach indicated an error rate of less than 0.7 mean dE_{CMC}. The XGBoost (linear model) 356 indicated a similar error rate as the Elastic Net 354 model. However, the use of the XGBoost (tree model) 358 in the Hybrid Model approach indicated a much improved error rate of less than 0.5 mean dE_{CMC}; while the Deep Learning 360 model indicated an error rate of just greater than 0.55 mean dE_{CMC}. As indicated by the graph 350, the XGBoost (tree model) 358 used in the Hybrid approach may provide about a thirty-percent improvement over traditional modeling (e.g., 304), or machine learning color models alone (e.g., 110, or 309, 313).

FIG. 3C is a graphical representation 360 of a color prediction error performance compared to a size of a database of color prediction data. That is, the rate of color prediction error 362 is compared with the amount of color prediction data 364 in a database, such as used for training a model or used for an analytical model. As illustrated, the error rate performance of a traditional analytical model 366 (e.g., K-M) shows a relationship of a slight error rate performance improvement as the amount of data increases. Further, a typical machine learning model 368 shows a much higher exponential improvement of the error rate performance, where a small database provides error rates higher than the traditional model 366, but far superior error rates as the amount of color prediction data increases. Additionally, the hybrid or combined traditional and machine learning model 370 show an exponential improvement in error rate performance that is at least equal to or greater than either the traditional model 366 and machine learning model 368 alone. The combined or hybrid model 370 has an error rate at least equal to or greater than the traditional model 366 when there is little color prediction data, which should be expected as one of the inputs to the hybrid model 370 is the output of the traditional model 366. However, the combined model shows marked improvement in error rate performance the machine learning model 368 is provided more data from which to train the color predictions, and the combination show improved error rate performance over the single models 366, 368 along all point of the graphical representation.

As described herein, the hybrid approach is configured to reduce a visual difference between a measured color (e.g., using a spectrophotometer) and the predicted color of the formulation. There are many color difference equations known in the art that can be used to describe color. One example is the dE_{CMC} color distance equation (e.g., as defined by the Society of Dyers and Colorists). However, any of the various equations may be appropriate for measuring the performances of the methods described herein, as it accounts for the color sensitivities of the human visual system. For example, for each of the compared models, the performance can be measured as the calculated average (mean) of dE_{CMC} for the color predictions under a standard illuminant with weighting factors 1=1.5 and c=1, between the two CIE Lab distance after converting the reflectance values to the CIE Lab space. Additionally, the root mean squared error (RMSE) for the prediction results can be provided. Below, is the dE_{CMC} equation [1] for calculating the distance between the two CIE Lab colors of the measured color (L1, a1, b1) and the predicted color (L2, a2, b2).

$${dE}_{CMC} = \sqrt{{( \frac{Δ L}{{IS}_{L}} )}^{2} {( \frac{Δ {C}_{ab}}{{cS}_{c}} )}^{2} + {( \frac{Δ {H}_{ab}}{{S}_{H}} )}^{2}}$$

where:

$$\begin{matrix}dC = {C}_{1} - {C}_{2} , {C}_{1} = \sqrt{{a}_{1}^{2} + {b}_{1}^{2} ,} {C}_{2} = \sqrt{{a}_{2}^{2} + {b}_{2}^{2}} \\ dH = \sqrt{{da}^{2} + {db}^{2}} - \overline{{dC}^{2}} \\ dL = {L}_{1} - {L}_{2} , da = {a}_{1} - {a}_{2} , db = {b}_{1} - {b}_{2} \\ {S}_{L} = \{ \begin{matrix}0.511 & if {L}_{1} < 16 \\ \frac{0.040975 {L}_{⊥}}{1 + 0.01765 {L}_{1}} & if {L}_{1} \geq 16\end{matrix} \\ {S}_{C} = \frac{0.0638 {C}_{1}}{1 + 0.0131 {C}_{1}} + 0.638 \\ {S}_{H} = {S}_{C} ( FT + 1 - F ) \\ T = \{ \begin{matrix}0.56 + \vert 0.2 cos ( {H}_{1} + 168 ° ) \vert & if 164 ° \leq {H}_{1} \leq 345 ° \\ 0.36 + \vert 0.4 cos ( {H}_{1} + 35 ° ) \vert & otherwise\end{matrix} \\ F = \sqrt{\frac{{C}_{1}^{4}}{{C}_{1}^{4} + 1900}} \\ H = arctan ( \frac{{b}_{1}}{{a}_{1}} ) \\ {H}_{1} = \{ \begin{matrix}H & if H \geq 0 \\ H + 360 ° & otherwise\end{matrix}\end{matrix} \qquad Equation 1$$

As described above, along with traditional, computer-based analytical models (e.g., K-M based model), machine learning models can be used to predict the color of a product, for a particular application. Various machine learning models may be utilized for the hybrid approach to color prediction, described herein. Further, various types of color prediction can be undertaken using this method. For example, a prediction of spectral reflectance of a coating on a target application may be the result of the hybrid approach for a target formulation. For example, Spectral Reflectance measures the amount of light reflected from a thin film over a range of wavelengths, with the incident light normal (perpendicular) to the sample surface. It is anticipated that other types of color measurement predictions may utilize the techniques described herein.

In some implementations, an Elastic Net machine learning model may be utilized. Elastic Net is a type of regularized regression model which provides a middle ground between Ridge regression and Lasso regression. Elastic Net uses a regularization term which is a simple mix or both Ridge and Lasso which is shown in the equation [2] below:

$$J ( θ ) = MSE ( θ ) + r α {\sum }_{i = 1}^{n} \vert {θ}_{i} \vert + \frac{1 - r}{2} α {\sum }_{i = 1}^{n} {θ}_{i}^{2} \qquad Equation 2$$

In this equation, the r is a regularization mix ratio, when r=0, Elastic Net is equivalent to Ridge regression, whereas when r=1, it is equivalent to Lasso regression. The elastic net may be tuned by searching for the optimal parameter values by a using a grid search with a 3 fold-cross validation on the training set in the search ranges shown in table I, below. A final model can then be fitted on the entire training set data.

Table I

| ENet parameters | Parameter Search Range |
|---|---|
| ′lambda′ | [0.001-100] |
| ′L1/L2 ratio′ | [0.1-1] |

### Parameter Search Grid for Elastic Net Algorithm

In some implementations, an extreme Gradient Boosting (XGBoost) machine learning with a linear model may be utilized. In the linear model, an XGBoost regressor algorithm can be tuned with the squared error objective which uses boosted linear models. In one example, thirty-one individual XGBoost models were tuned with a grid search to find the optimal configurations for the learning rate, estimator numbers, lambda and alpha parameters using a threefold-cross validation on the training set. After the optimal parameters were found, the final models were fitted on the entire training set data. Table II, below, shows the searched ranges for the Linear Regression Booster algorithm.

TABLE II

| XGBoost (linear) parameters | Parameter Search Range |
|---|---|
| learning rate | [0.05-0.2] |
| n estimators | [500-2500] |
| reg lambda | [0.1-100] |
| alpha | [0.1-100] |

### Parameter Search Grid for XGBoost (Linear) Algorithm

In some implementations, an extreme Gradient Boosting (XGBoost) machine learning with a tree model may be utilized. In the tree model, the eXtreme Gradient Boosting regressor algorithm, which uses boosted tree models, can be tuned with the squared error objective. Similar to the boosted linear implementation, in one example, thirty-one individual XGBoost models can be tuned with a grid search to find the optimal configurations for the parameters for learning rate, max depth, subsample rate, min child weight, number of estimators, and column sample by node, and column and sample by tree, for the search ranges shown in Table III, below. Once the optimal parameters were found, a final model can be fitted on the entire training set.

TABLE III

| XGBoost (tree) parameters | Parameter Search Range |
|---|---|
| learning rate | [0.05-0.2] |
| max depth | [5-20] |
| subsample | [0.5-1] |
| min child weight | [0.5-1] |
| n estimators | [500-2500] |
| colsample bynode | [0.33-I] |
| colsample by tree | [0.33-1] |

### Parameter Search Grid for XGBoost (Tree) Algorithm

In some implementations, a Deep Learning (DL) machine learning model may be utilized. In this model, multilayer perceptron (MLP) model architectures, including a fully-connected feed-forward model, a Resnet skip-connection model, and a wide-and-deep-learning like model architectures can be utilized. In various configurations, the network architecture may be a single model which has fixed inputs nodes which is equal to the size of the variables used, for example, and thirty-one output nodes. In this implementation, different configurations can be searched for the optimal number of layers, hidden node numbers and hidden node activations, which included the ‘sigmoid’, ‘tanh’, ‘relu’, ‘elu’, ‘selu’ and ‘softplus’ activations. The following list gives brief descriptions of the above mentioned activations:

The Logistic Sigmoid is an activation function for neural networks which transforms an input into a value between 0 and 1.

$$sigmoid ( x ) = \frac{1}{1 + exp ( - x )} \qquad Equation 3$$

The Hyperbolic tangent (Tanh), has a similar shape to the Logistic Sigmoid and is a function that outputs values between −1 and 1.

$$tan h ( x ) = \frac{{e}^{x} - {e}^{- x}}{{e}^{x} + {e}^{- x}} \qquad Equation 4$$

The ReLU (Rectified Linear Unit) activation is a non-linear function that returns the input value input directly, or the value 0 if the input value is 0 or less.

$$ReLU ( x ) = \{ \begin{matrix}0 & if x > 0 \\ x & if x \leq 0\end{matrix} \qquad Equation 5$$

SELU and ELU are both variants of the ReLU activation function given by the 2 following equations:

$$SELU ( x ) = λ \{ \begin{matrix}1 & if x > 0 \\ α {e}^{x} & if U \leq 0\end{matrix} \qquad Equation 6$$

where α and λ are constants 1.6732 and 1.0507 respectively for standard scaled inputs, and

$$ELU ( x ) = \{ \begin{matrix}x & if x > 0 \\ α ( {e}^{x - 1} ) & if x < 0\end{matrix} \qquad Equation 7$$

where value for alpha is picked typically between 0.1 and 0.

Finally, the softplus is a smooth approximation of the rectifier activation function.

$$Softplus ( a ) = ln ( ( 1 + {e}^{a} ) \qquad Equation 8$$

The Deep Learning model can be tuned using the Adam optimizer to minimize various loss functions such as the MSE, MAE and Huber loss. The Huber loss can be used in particular to prevent the potential impact of outliers which may still be present in the data. The equation [8] for Huber loss is given below.

$${L}_{δ} = \{ \begin{matrix}\frac{1}{2} {( y - f ( x ) )}^{2} & for \vert y - f ( x ) \vert \leq δ \\ δ ( \vert y - f ( x ) \vert - \frac{1}{2} δ ) , & otherwise\end{matrix} \qquad Equation 9$$

The DL networks can be trained to minimize the losses up to a maximum of two-thousand epochs. In addition, to control model complexity, the weights regularization of L2 penalty applied to the hidden layer weights and an early stopping criteria can be used to find the appropriate training length by observing if a validation loss did not improve after one-hundred epochs, based on a validation set which was randomly selected from ten percent of the training set population. Table IV, below, shows the searched ranges for parameter in the DL algorithm.

TABLE IV

| Deep Learning Parameters | Parameter Search Range |
|---|---|
| number of layers | [I-5] |
| hidden node size | [50-500] |
| loss functions | [MSE, MAE, Huber ] |
| learning rate | [0.001-0.1] |
| L2 regularization | [0.000-0.1] |
| hidden activations | [sigmoid, tanh, relu, elu, selu, softplus] |

### Parameter Search Grid for Deep Learning

In one implementation, a selected dataset (e.g., comprising sample formulations and resulting color measurements) can be divided into a training set (e.g., ninety percent of the dataset) and a sample set (e.g., ten percent of the selected dataset). The training set can be used to train and build the predictive models, while the sample set can be used as a testing set to evaluate the performance of the trained models. In some implementations, to investigate stability of the performance of the models the results can be evaluated by running a Monte Carlo simulation for the various results, by repeating the process of randomly splitting the selected dataset into training and sample sets as described above, rebuilding the models, and evaluating them. This may allow for a variance in model performances to be inspected. For a Monte Carlo Simulation, the average and the standard deviation (SD) of the performances can be provided for each performance measure.

TABLE V

| Machine Learning Methods | dE_{CMC} | RMSE |
|---|---|---|
| Elastic Net | 0.675 | 1.366 |
| Extreme Gradient Boosting (Linear) | 0.674 | 1.326 |
| Extreme Gradient Boosting (Trees) | 0.479 | 1.057 |
| Multilayer Perceptions (DL) | 0.539 | 1.111 |

### Evaluation of Performance for Various Models

As described herein, the hybrid approach to color prediction for a formulation of a coating material can provide an improved performance over either the traditional analytical model or the machine learning model. For example, prediction of spectral reflectance from colorant concentration values using a dataset of paints formulations and the hybrid approach of an analytical (e.g., Kubelka-Munk) model and Machine Learning methods involves using the Machine Learning approach to correct the initial prediction made by the analytical model. This method can involve optimizing the Machine Learning models to predict residuals of the analytical model's predictions to the measured spectral reflectance values of the color formulations being predicted. As described herein, various machine learning methods may be used, for example, Elastic Net, two types of eXtreme Gradient Boosting Algorithms (linear and tree), and a Deep Learning model.

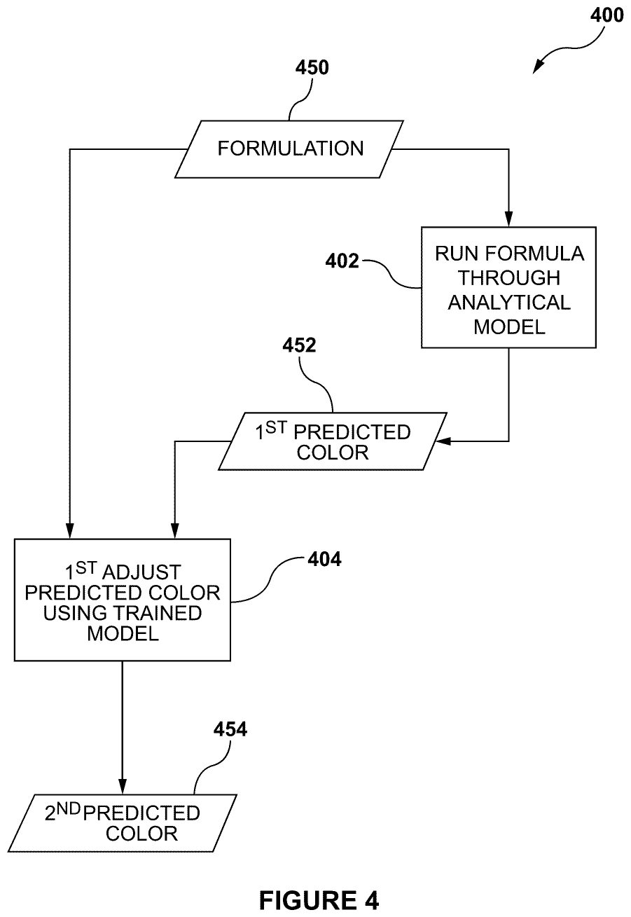

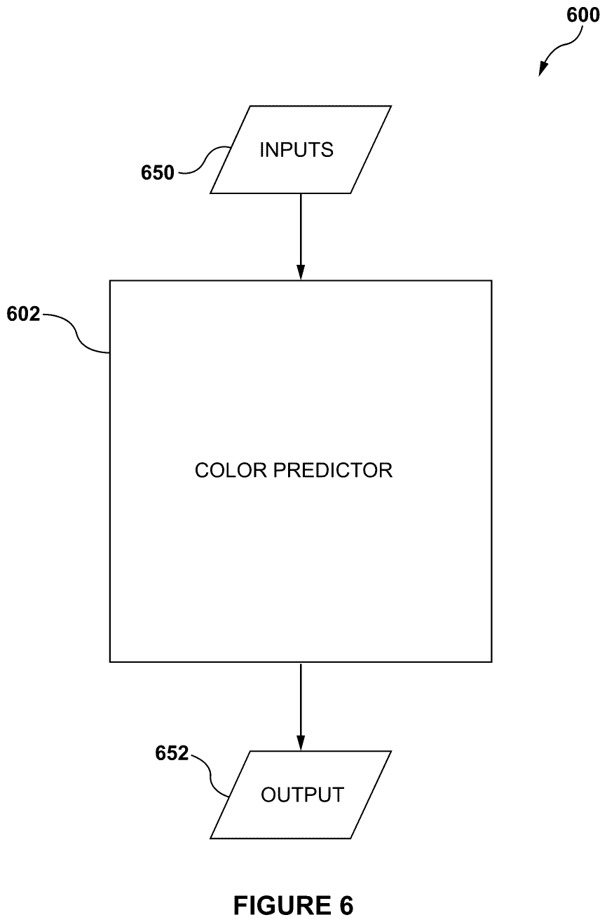

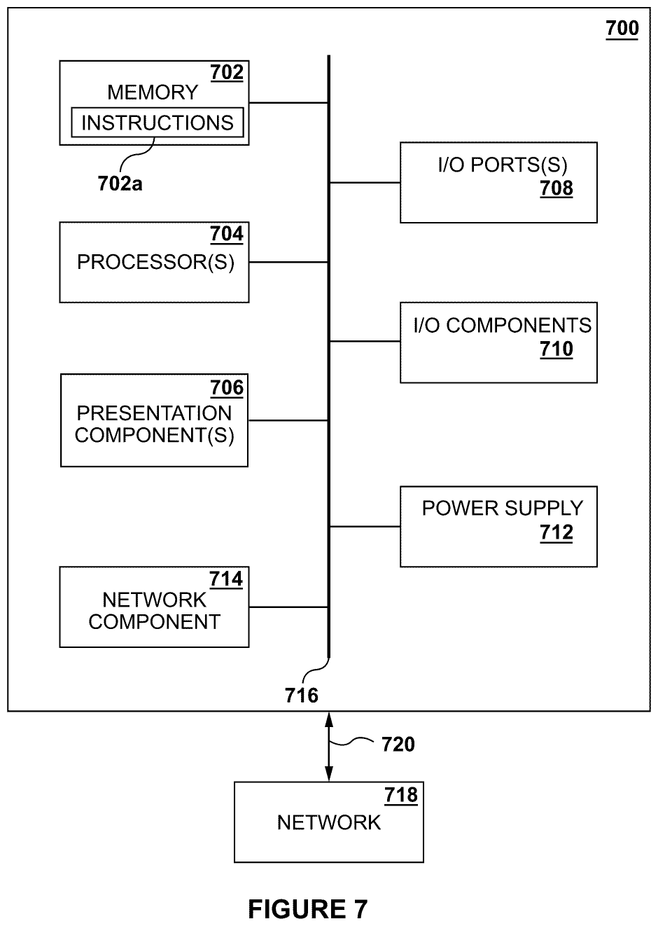

As described herein, a method may be devised that provides for improved prediction of a color from a given formulation for a surface coating material (e.g., paint). FIG. 4 is a flow diagram that illustrates an exemplary method 400 for predicting a color from a formulation. In some implementations, the method 400 may be performed by a color predictor system (e.g., 600 of FIG. 6 below), and/or a computing device (e.g., 700 illustrated in FIG. 7 below, which may form part of or implement part of the color predictor system 600).

In the example method 400, at 402, a formulation 450 for a surface coating is run through an analytical model. In some implementations, the formulation 450 and the target application for the coating can be run through the analytical model. For example, the type of application may affect the perceived color of the surface coating cured on a surface. The type of application may include the type of surface, such as wood siding, metal vehicle fender, vinyl sheet, etc. Further the type of application may include the method of application, such as spray applied, screen printed, brush or roller applied, etc. Additionally, the type of application may include the type of finish of the coating, such as high gloss, gloss, matt, flat, etc. In these implementations, data indicative of the type of application (e.g., including each known variable-surface, application, finish, and others) can be an input to the analytical model along with the formulation. With regard to the formulation 450, the formulation of the formulation can include any appropriate ingredient for the type of surface coating, along with the amount of the respective ingredients. For example, coatings often include various binders, pigments (e.g., colorants), effect elements, preservatives, hardeners, curing agents, flow modifiers, and other additives used for a coating. In this example, any one or more of these ingredients can be included in the formulation 450 along with the corresponding amount, or combined into groups of ingredients along with the groups' amount. Often, formulations are expressed as groups of data, which can include ingredients and characteristics of the grouped data, such as binder technology data, pigment (colorant) load data, pigment (colorant) class data, and application method.

As one example, an analytical model can comprise a computer-based model that uses an algorithm to identify an expected color expressed as light reflectance from a surface. As one example, a computer-based Kubelka-Munk analytical model (K-M Model) uses the Kubelka-Munk theory to predict color reflectance on a surface. The Kubelka-Munk theory describes the spectral power distribution of light emerging from an illuminated surface (e.g., with light source perpendicular to the surface) of opaque or translucent media as a result of reflection, scattering and partial absorption, often used for paint films.

Returning to the method 400 of FIG. 4, the result of running the formulation 450 through the analytical model is a first predicted color 452, that is, data indicative of a predicted color. That is, for example, an initial (first) prediction of the spectral reflectance can be performed using a K-M model for the formulation from colorant concentration values. In some implementations, the formulation can be iterated through the K-M Model resulting in a first prediction comprising a set of analytical model predictions for the formulation. At 404 in the exemplary method 400, the first predicted color is adjusted using a trained machine learning color model, resulting in a second predicted color. That is, the first predicted color and the formulation (e.g., and application) are input to the trained machine learning color model, producing the second predicted color as an output.

As an example, the trained machine learning color model is used to correct the initial prediction made in step 402, such that the resulting prediction is closer to a measured spectral reflectance for the formulation and application. For example, this may be achieved by optimizing the trained machine learning color model to predict the residuals of the K-M model's predictions to the measured spectral reflectance values of the color formulations being predicted. In some implementations, the inference of new data-points of the K-M model's predictions are summed up with the trained machine learning color model's prediction. In one example, for the step 404 including the trained machine learning color modelling, the inputs to the models can comprise a concatenation of all the vectors in the input variables and the output (targets variables or the second color prediction) are the residuals between the estimation made initially by the K-M model and the measured spectral reflectance values of each of thirty-one wavelengths. The input variable can comprise the coating formulation, which are the concentrations of forty-one different toners by their percentage composition values—a sparse vector of length forty-one, where each element represents the concentration amount of a toner (e.g., where a value of zero indicates that the toner is not in use). As described herein, the term “toners” may describe either a colorant or a binder. Further, the input variables can comprise the spectral reflectance of the K-M model's predictions—a vector of length thirty-one for each value in the spectral reflectance curve or a set of multiple spectra for effect coatings like automotive metallic paint. In this example, the target variable (e.g., second color prediction) can comprise the measured spectral reflectance values of the resulting color from the formulation mix—a vector of length thirty-one. It should be noted that, while the most common spectrophotometers use a vector of thirty-one data points, as illustrated in this example, there are exceptions. In some embodiments, there may be a vector of length fifty-five (55), which corresponds to the number of unique toners. In one embodiment, the product identification acts as an identifier of the coating product previously defined from which the sample originated from a “one-hot vector of length,” which corresponds to the number of products in a particular data set. The term “one-hot vector of length” may be used with categorical data. Because many machine learning models need their input variables to be numeric, categorical variables need to be transformed in the pre-processing part and applied to nominal variables in order to improve the performance of the algorithm. In one embodiment, the one-hot vector length is 18. Other one-hot vector lengths are also contemplated. The techniques and methods described here can also apply to wavelengths that are outside the visual spectrum (e.g., NIR-UV). In some embodiments, a layer thickness which describes the thickness of the coating applied, which is referred to as a thickness scalar (expressed as μm). In many embodiments, the thickness scalar is 1.0 μm. Other thickness scalars such as 0.9 μm and others may be contemplated. In some embodiments, an undercoat may be used with the coating product. In some embodiments, the undercoat has a vector of 31. Other vectors are also contemplated.

In many embodiments, the ANN product independent model may provide: 1) colorant recipes, 2) undercoat, 3) at least one coating product, 4) thickness scalar, or combinations thereof. In many embodiments, the trained machine learning color prediction model is further trained over a set of application methods comprising techniques for applying the coating formulation to a surface, adding an undercoat, specifying a coating product, specifying a thickness scalar, or combinations thereof.

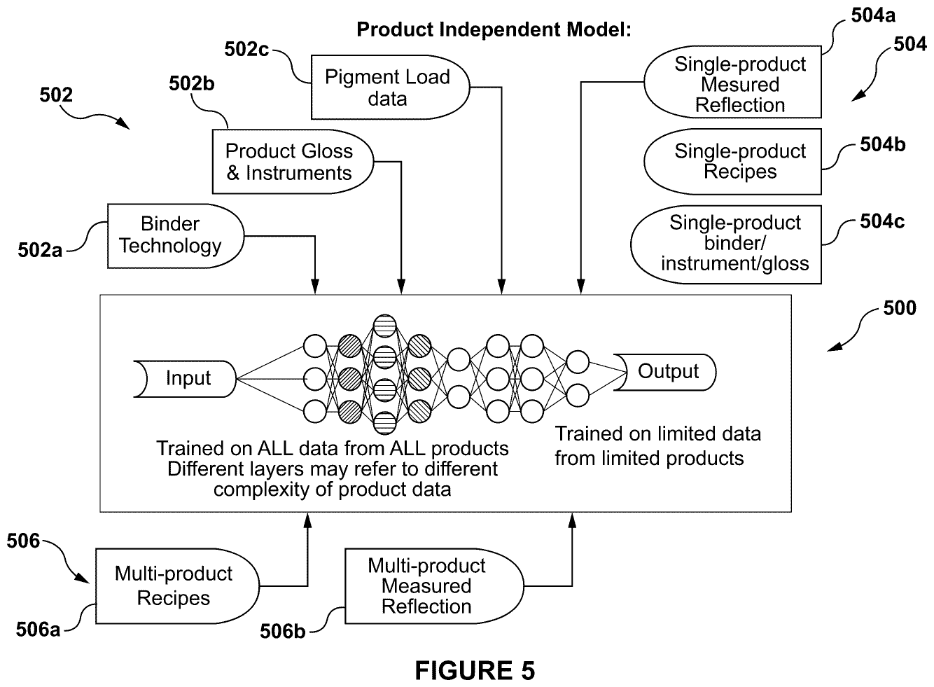

FIG. 5 is a diagram illustrating another example implementation of a color predictor model 500, for example, which may be implemented in a color predictor system (e.g., such as system 600 of FIG. 6, below). In this example, the color predictor model 500 is a product independent model and a possible embodiment of 250 in FIG. 2. That is, for example, the model is trained with data sets that are from existing (e.g., any) coatings products. In this example, the product independent model can be accurately applied to products that did not provide training data (e.g., or only provided a limited number of training data). In this implementation, a multi-layered deep-learning algorithm can be used, where separate layers 502a-c, 504a-c, 506a-b, or sets of layers 502, 504, 506 can have a dedicated function. Some layers or set of layers 502a-c, 504a-c, 506a-b, 502, 504, 506 can be trained on all the myriad colorant/coating data that is available, as described above, and some layers or set of layers 502a-c, 504a-c, 506a-b, 502, 504, 506 can be trained on a small subset of the available data.

In this implementation, as illustrated, some of the layers can include: binder technology 502a, including the different types of materials, chemistry, and mixing techniques; product gloss and instruments 502b, including measurements of gloss and instruments for measuring; and pigment load and other pigment data 502c, including measurement of load and type(s) of pigment; in a set of layers 502. Further, layers can include: single-product measured reflectance 504a, for respective products; single product recipes 504b, for each product; and single-product binder/instrument/gloss 504c data for each product; in a set of layers 504. Additionally, the layers can include: multi-product recipes 506a, for a product line; and multi-product measured reflectance 506b, for a product line; in a set of layers 506.

In this implementation, the model 500 may have some of the layers 502, 504, 506 trained on all data from all products or product lines that may the subject of color matching prediction. In this way, for example, there is a single-color prediction model that is suitable to predict colors for all products, independent of a particular product. As an example, the color behavior of a select number of pigments can be determined, independent of the product parameters (e.g., binder chemistry, additives, production methods, application, and gloss level). The result is that when a product is developed, based on the ingredients, the production method and the application method, the color model can be identified without the need to first create sample colors (e.g., or a very limited number of sample colors that are used to train the last layer). As another example, a second advantage is that when a color is made for a certain product, it can be translated with high accuracy to another product, which means that identification of other colors may no longer be required.

As an example, as illustrated in FIG. 6, a color predictor system 600 can be used to more accurately predict the color of a coating formulation. In some implementations, the color predictor system 600 can comprise computer implemented components such as the analytical model (e.g., K-M Model, such as 104 in FIG. 1) and the trained learning engine color model (e.g., 124 of FIG. 1). Further, the color predictor system 600 can comprise at least one processor to process data, and memory to store instructions and data used to implement the models, as described below in FIG. 6. Input data 650 is input to the color predictor system 600, where the input 650 can comprise data indicative of the formulation, as described herein. Output data 652 is output from the color predictor system 600, where the outputs 652 can comprise data indicative of a predicted color for the formulation (e.g., and application), as described herein.

In some examples, one or more algorithms (e.g., disposed as instructions and/or programming) stored in memory, are used by the color predictor system 600 to predict or estimate the color of the formulation for the surface coating application. For example, the color predictor system 600 is configured to use combinational or computational algorithmic logic to process the inputs 650, and optionally any additional inputs, to predict or estimate the color for the surface coating application, as an output 652. In one example, empirical, experimental or simulation data is used to train or configure portions of the color predictor system 600, and then the inputs 650, and optionally the additional inputs, are processed to predict or estimate the color the surface coating application. In some examples, machine learning is used to train or configure the color predictor system 600 based on a training data from simulations or feedback received from previously predicted formulations and resulting colors to a more accurate result. In some examples, artificial intelligence (AI) is used as part of the training and/or processing of the color predictor system 600 to converge to a more accurate result. Further, ongoing training can be used to periodically update the prediction results of the combinational or computational algorithmic logic.

With reference now to FIG. 7, a block diagram of the computing device 700 suitable for implementing various aspects of the disclosure is described (e.g., a monitoring system). FIG. 7 and the following discussion provide a brief, general description of a computing environment in/on which one or more or the implementations of one or more of the methods and/or system set forth herein may be implemented. The operating environment of FIG. 7 is merely an example of a suitable operating environment and is not intended to suggest any limitation as to the scope of use or functionality of the operating environment. Example computing devices include, but are not limited to, personal computers, server computers, hand-held or laptop devices, mobile devices (such as mobile phones, mobile consoles, tablets, media players, and the like), multiprocessor systems, consumer electronics, mini computers, mainframe computers, distributed computing environments that include any of the above systems or devices, and the like.

Although not required, implementations are described in the general context of “computer readable instructions” executed by one or more computing devices. Computer readable instructions may be distributed via computer readable media (discussed below). Computer readable instructions may be implemented as program modules, such as functions, objects, Application Programming Interfaces (APIs), data structures, and the like, that perform particular tasks or implement particular abstract data types. Typically, the functionality of the computer readable instructions may be combined or distributed as desired in various environments. For example, artificial intelligence solutions often comprise a client and server side implementation, where the implemented software program or web-application could be installed on the client side. Further, in this example, the calculations may be performed on the server side, and communication takes place using one of the aforementioned protocols.

In some examples, the computing device 700 includes a memory 702, one or more processors 704, and one or more presentation components 706. The disclosed examples associated with the computing device 700 are practiced by a variety of computing devices, including personal computers, laptops, smart phones, mobile tablets, hand-held devices, consumer electronics, specialty computing devices, etc. Distinction is not made between such categories as “workstation,” “server,” “laptop,” “hand-held device,” etc., as all are contemplated within the scope of FIG. 7 and the references herein to a “computing device.” The disclosed examples are also practiced in distributed computing environments, where tasks are performed by remote-processing devices that are linked through a communications network. Further, while the computing device 700 is depicted as a single device, in one example, multiple computing devices work together and share the depicted device resources. For instance, in one example, the memory 702 is distributed across multiple devices, the processor(s) 704 provided are housed on different devices, and so on.

In one example, the memory 702 includes any of the computer-readable media discussed herein. In one example, the memory 702 is used to store and access instructions 702a configured to carry out the various operations disclosed herein. In some examples, the memory 702 includes computer storage media in the form of volatile and/or nonvolatile memory, removable or non-removable memory, data disks in virtual environments, or a combination thereof. In one example, the processor(s) 704 includes any quantity of processing units that read data from various entities, such as the memory 702 or input/output (I/O) components 710. Specifically, the processor(s) 704 are programmed to execute computer-executable instructions for implementing aspects of the disclosure. In one example, the instructions 702a are performed by the processor 704, by multiple processors within the computing device 700, or by a processor external to the computing device 700. In some examples, the processor(s) 704 are programmed to execute instructions such as those illustrated in the flow charts discussed herein and depicted in the accompanying drawings.

In other implementations, the computing device 700 may include additional features and/or functionality. For example, the computing device 700 may also include additional storage (e.g., removable and/or non-removable) including, but not limited to, magnetic storage, optical storage, and the like. Such additional storage is illustrated in FIG. 7 by the memory 702. In one implementation, computer readable instructions to implement one or more implementations provided herein may be in the memory 702 as described herein. The memory 702 may also store other computer readable instructions to implement an operating system, an application program and the like. Computer readable instructions may be loaded in the memory 702 for execution by the processor(s) 704, for example.

The presentation component(s) 706 present data indications to an operator or to another device. In one example, the presentation components 706 include a display device, speaker, printing component, vibrating component, etc. One skilled in the art will understand and appreciate that computer data is presented in a number of ways, such as visually in a graphical user interface (GUI), audibly through speakers, wirelessly between the computing device 700, across a wired connection, or in other ways. In one example, the presentation component(s) 706 are not used when processes and operations are sufficiently automated that a need for human interaction is lessened or not needed. I/O ports 708 allow the computing device 700 to be logically coupled to other devices including the I/O components 710, some of which is built in. Implementations of the I/O components 710 include, for example but without limitation, a microphone, keyboard, mouse, joystick, pen, game pad, satellite dish, scanner, printer, wireless device, camera, etc.

The computing device 700 includes a bus 716 that directly or indirectly couples the following devices: the memory 702, the one or more processors 704, the one or more presentation components 706, the input/output (I/O) ports 708, the I/O components 710, a power supply 712, and a network component 714. The computing device 700 should not be interpreted as having any dependency or requirement related to any single component or combination of components illustrated therein. The bus 716 represents one or more busses (such as an address bus, data bus, or a combination thereof). Although the various blocks of FIG. 7 are shown with lines for the sake of clarity, some implementations blur functionality over various different components described herein.

The components of the computing device 700 may be connected by various interconnects. Such interconnects may include a Peripheral Component Interconnect (PCI), such as PCI Express, a Universal Serial Bus (USB), firewire (IEEE 1394), an optical bus structure, and the like. In another implementation, components of the computing device 700 may be interconnected by a network. For example, the memory 702 may be comprised of multiple physical memory units located in different physical locations interconnected by a network.

In some examples, the computing device 700 is communicatively coupled to a network 718 using the network component 714. In some examples, the network component 714 includes a network interface card and/or computer-executable instructions (e.g., a driver) for operating the network interface card. In one example, communication between the computing device 700 and other devices occurs using any protocol or mechanism over a wired or wireless connection 720. In some examples, the network component 714 is operable to communicate data over public, private, or hybrid (public and private) connections using a transfer protocol, between devices wirelessly using short range communication technologies (e.g., near-field communication (NFC), Bluetooth® branded communications, or the like), or a combination thereof.

The connection 720 may include, but is not limited to, a modem, a Network Interface Card (NIC), an integrated network interface, a radio frequency transmitter/receiver, an infrared port, a USB connection or other interfaces for connecting the computing device 700 to other computing devices. The connection 720 may transmit and/or receive communication media.

Although described in connection with the computing device 700, examples of the disclosure are capable of implementation with numerous other general-purpose or special-purpose computing system environments, configurations, or devices. Implementations of well-known computing systems, environments, and/or configurations that are suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, VR devices, holographic device, and the like. Such systems or devices accept input from the user in any way, including from input devices such as a keyboard or pointing device, via gesture input, proximity input (such as by hovering), and/or via voice input.

Implementations of the disclosure are described in the general context of computer-executable instructions, such as program modules, executed by one or more computers or other devices in software, firmware, hardware, or a combination thereof. In one example, the computer-executable instructions are organized into one or more computer-executable components or modules. Generally, program modules include, but are not limited to, routines, programs, objects, components, and data structures that perform particular tasks or implement particular abstract data types. In one example, aspects of the disclosure are implemented with any number and organization of such components or modules. For example, aspects of the disclosure are not limited to the specific computer-executable instructions or the specific components or modules illustrated in the figures and described herein. Other examples of the disclosure include different computer-executable instructions or components having more or less functionality than illustrated and described herein. In implementations involving a general-purpose computer, aspects of the disclosure transform the general-purpose computer into a special-purpose computing device when configured to execute the instructions described herein.

By way of example and not limitation, computer readable media comprises computer storage media and communication media. Computer storage media include volatile and nonvolatile, removable, and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals per se. In one example, computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium used to store information for access by a computing device. In contrast, communication media typically embody computer readable instructions, data structures, program modules, or the like in a modulated data signal such as a carrier wave or other transport mechanism and include any information delivery media.

While various spatial and directional terms, including but not limited to top, bottom, lower, mid, lateral, horizontal, vertical, front and the like are used to describe the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

### EXAMPLES

The data originates from a commercial database of paint colorant recipes used for coatings. The database includes paint colorant recipes for 4150 color which belong to 18 distinct products, where each product contains between 170 and 220 paint colorant recipes and are not included in equal quantity. They are produced from mixing no more than 4 out of a set of 55 different kinds of colorants/binders (e.g., of them 37 are colorants and 18 are binders). The paint colorant recipes have overlapping use of colorants between product but, each products uses a binder unique to one product only. The corresponding spectral reflectance curve is measured by a Spectrophotometer. The measurements of the reflectance spectra consist of the visual spectrum in the range of 400 to 700 nanometers at 10 nanometer step intervals, 31 in total, using a D/8 integrating sphere instrument. The reflectance values are therefore vectors of length 31 which correspond to each wavelength for the measured range of the spectral curve. All variables in the data are numerical and continuous, and thus this work is a regression task of predicting 31 target variables. For the data below, the dE CMC formula which is used to approximate the color sensitivities of the human visual system and measured as the calculated average of dE CMC (1.5, 1) for the test set predictions under a reference illuminant with weighting factors 1=1.5 and c=1. Also provided is the root mean squared error for the prediction results.

The method for color matching described herein, also referred to as Feed-Forward ANN Product independent model or ANN Product independent model, was tuned using the Adam optimizer to minimize Huber loss. The ANN Product independent model was trained to minimize the losses up to a maximum of 2000 epochs. In addition, to control model complexity, the weights regularization of L1 and L2 penalty applied to the hidden layer weights of the ANN Product independent model and an early stopping criteria was used to the find appropriate training length by observing if a validation loss did not improve after 200 epochs based on a validation set which was randomly selected from 10 percent of the training set population.

$$λ {\sum }_{j = 0}^{M} \vert {W}_{j} \vert \qquad Equation 9$$

For both Equations 9 and 10, Wj is a single weight whereas the ANN Product independent model uses multiple weights (W).

$$λ {\sum }_{j = 0}^{M} {W}_{j}^{2} \qquad Equation 10$$

The ANN Product independent model was trained on a 90 and 10 percent split for train and test set respectively, by selecting 10 percent from each product, in order to provide data sets for both training and testing for more unbiased and improved estimates of the performance in the model.

TABLE VI
Prediction Performance
Comparisons of Product independent models

| Model | RMSE | dE CMC |
|---|---|---|
| K-M single model for 18 products | NA | 0.80 |
| K-M Product independent model | NA | 2.00 |
| ANN Product independent model | 4.92 | 1.33 |

Table V1 provides comparison of performance for the ANN Product independent model; a benchmark implementation of a traditional K-M analytical model which was modelled individually and separately for each single product and an implementation of a product independent K-M analytical model. As seen from Table VII below, while the performance of the traditional K-M analytical model is stable (dE CMC error of 0.8), the performance of a K-M product independent analytical model presents significant challenges in modelling a dataset with 18 products together which contain several binders in the dataset which analytical models are inept at handling by design, resulting in an average dE CMC error of 2. In comparison the method described herein provides a dE CMC of 1.33 and demonstrates that the ANN Product independent model approach may be able to learn from an unconventionally prepared dataset for color prediction.

TABLE VII
Breakdown of Prediction Performance Comparisons per product

| | Model and perdition | | |
|---|---|---|---|
| | Performance in dE CMC | | |
| | | ANN | |
| | K-M | Product | K-M |
| | Product | Independent | Product |
| | Independent | Model | Independent |
| Products | Model | (Ours) | Model |
| Product 1 | 0.605 | 0.902 | 1.629 |
| Product 2 | 1.145 | 1.004 | 2.316 |
| Product 3 | 0.541 | 0.315 | 2.334 |
| Product 4 | 0.512 | 0.589 | 2.762 |
| Product 5 | 0.963 | 0.692 | 3.033 |
| Product 6 | 0.664 | 0.745 | 1.886 |
| Product 7 | 0.899 | 0.860 | 1.918 |
| Product 8 | 0.842 | 0.914 | 1.965 |
| Product 9 | 0.676 | 0.504 | 1.865 |
| Product 10 | 0.640 | 0.841 | 2.164 |
| Product 11 | 0.788 | 0.798 | 1.676 |
| Product 12 | 0.581 | 3.776 | 1.887 |
| Product 13 | 1.599 | 0.506 | 1.963 |
| Product 14 | 0.618 | 0.404 | 1.733 |
| Product 15 | 0.840 | 2.912 | 1.682 |
| Product 16 | 0.180 | 0.425 | 8.530 |
| Product 17 | 0.756 | 0.533 | 1.972 |
| Product 18 | 1.465 | 1.235 | 1.859 |

Table VII above provides a breakdown of the prediction performance in average dE CMC for each product. In particular, the ANN product independent model performs better than all cases of K-M product independent models. When comparing performance for each product, the ANN product independent model performs comparable to the traditional K-M analytical models and performs better color predictions for 9 out of 18 products. However, there may be some outliers in the performance of the ANN product independent model for a few product which likely affects the overall average performance (particularly product 12 and 15) which may be caused by imbalance in product samples of training data.

### EXAMPLE EMBODIMENTS

Embodiment 1—One embodiment for color matching to a coating formulation, can comprise the steps of: inputting a coating formulation into a trained machine learning color prediction model, where the coating formulation comprises a binder component selected from a set of binder components and a colorant component selected from a set of colorant components, resulting in a color match prediction for the coating formulation, wherein the trained machine learning color prediction model is trained over the set of binder components that are used in a plurality of coating formulations, and is trained over the set of colorant components that are used in a plurality of coating formulations, resulting in the trained machine learning color prediction model able to predict the color match for the plurality of coating formulations.

Embodiment 2—the method of embodiment 1, wherein the trained machine learning color prediction model is further trained over a set of application methods comprising techniques for applying the coating formulation to a surface, adding an undercoat, specifying a coating product, specifying a thickness scalar, or combinations thereof.

Embodiment 3—the method of embodiments 1 and 2, wherein set of colorant components comprises a plurality of pigments.

Embodiment 4—the method of embodiment 3, wherein the trained machine learning color prediction model is further trained over a set of pigment loads for respective pigments in the set of colorant components.

Embodiment 5—the method of embodiment 3, wherein the trained machine learning color prediction model is further trained over a set of pigment indices for respective pigments in the set of colorant components.

Embodiment 6—the method of embodiments 1-5, wherein the set of binder components comprise a merely binders for a target product line of coatings.

Embodiment 7—the method of embodiments 1-6, wherein respective binder components in the set of binder components is associated with merely one product in the target product line.

Embodiment 8—the method of embodiment 7, wherein the color component in the set of color components are utilized in the respective products of the product line.

Embodiment 9—the method of embodiments 1-8 wherein the trained machine learning color prediction model comprises an elastic net regression model.

Embodiment 10—the method of embodiments 1-9 wherein the trained machine learning color prediction model comprises a gradient boosting algorithm.

Embodiment 11—the method of embodiment 10 wherein the trained machine learning color prediction model comprises a gradient boosting algorithm tuned with linear parameters.

Embodiment 12—the method of embodiment 10 wherein the trained machine learning color prediction model comprises a gradient boosting algorithm tuned with tree parameter models.

Embodiment 13—the method of embodiments 1-9 wherein the trained machine learning color prediction model comprises a deep learning model architecture.

Embodiment 14—the method of embodiment 13, wherein the deep learning model architecture comprises a multilayer perceptron model architecture.

Embodiment 15—the method of embodiment 14, wherein the multilayer perceptron model architecture comprises a fully-connected feed-forward model, a Resnet skip-connection model, a wide-and-deep learning model, or combinations thereof.

Embodiment 16—the method of embodiments 13-15, wherein the deep learning model utilizes at least one parameter and wherein at least one parameter comprises number of layers, hidden node size, loss functions, learning rate, L2 regularization, hidden activations, or combinations thereof.

Embodiment 17—A coating prepared using the method of any of embodiments 1-16.

Embodiment 18—A method of creating a machine learning color prediction model that predicts a color of a coating based on a coating formulation, the method comprising: training a machine learning model over a set of binder components, wherein the binder components are found in a target product line of coatings; and training the machine learning model over a set of colorant components, wherein the colorant components are found in the target product line of coatings. Wherein the training results in a machine learning color prediction model that predicts the color of a coating from the target product line based at least on a formulation that comprises a binder component selected from a set of binder components and a colorant component selected from a set of colorant components.

Embodiment 19—the method of embodiment 18, wherein the trained machine learning color prediction model comprises an elastic net regression model.

Embodiment 20—the method of embodiments 18 and/or 19, wherein the trained machine learning color prediction model comprises a gradient boosting algorithm.

Embodiment 21—the method of embodiment 20, wherein the trained machine learning color prediction model comprises a gradient boosting algorithm tuned with linear parameters.

Embodiment 22—the method of embodiments 20, wherein the trained machine learning color prediction model comprises a gradient boosting algorithm tuned with tree parameter models.

Embodiment 23—the method of any embodiments 18-22, wherein the trained machine learning color prediction model comprises a deep learning model architecture.

Embodiment 24—the method of embodiment 23, wherein the deep learning model architecture comprises a multilayer perceptron model architecture.

Embodiment 25—the method of embodiment 24, wherein the multilayer perceptron model architecture comprises a fully-connected feed-forward model, a Resnet skip-connection model, a wide-and-deep learning model, or combinations thereof.

Embodiment 26—the method of any embodiments 23-25, wherein the deep learning model utilizes at least one parameter and wherein at least one parameter comprises number of layers, hidden node size, loss functions, learning rate, L2 regularization, hidden activations, or combinations thereof.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

Various operations of implementations are provided herein. In one implementation, one or more of the operations described may constitute computer readable instructions stored on one or more computer readable media, which if executed by a computing device, will cause the computing device to perform the operations described. The order in which some or all of the operations are described should not be construed as to imply that these operations are necessarily order dependent. Alternative ordering will be appreciated by one skilled in the art having the benefit of this description. Further, it will be understood that not all operations are necessarily present in each implementation provided herein.

Any range or value given herein can be extended or altered without losing the effect sought, as will be apparent to the skilled person.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

As used in this application, the terms “component,” “module,” “system,” “interface,” and the like are generally intended to refer to a computer-related entity, either hardware, a combination of hardware and software, software, or software in execution. For example, a component may be, but is not limited to being, a process running on a processor, a processor, an object, an executable, a thread of execution, a program and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device, carrier or media. Of course, those skilled in the art will recognize many modifications may be made to this configuration without departing from the scope or spirit of the claimed subject matter.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A method for predicting a color for a coating, comprising:
obtaining a first color prediction from an analytical model based on a formulation for the coating input to the analytical model;
obtaining an output from a trained machine learning model, wherein the formulation and the first color prediction from the analytical model are input to the trained machine learning model to generate the output; and
outputting a final color prediction for the coating based on at least one of the output from the trained machine learning model or the first color prediction.

2. The method of claim 1, wherein the formulation indicates at least one binder and at least one colorant.

3. The method of claim 1, wherein the output from the trained machine learning model is an adjustment to the first color prediction.

4. The method of claim 1, wherein the output is a residual of the first color prediction.

5. The method of claim 4, wherein the residual is added to the first color prediction to generate the final color prediction.

6. The method of claim 1, wherein the output is a plurality of residuals for values indicated by the first color prediction for a plurality of wavelengths.

7. The method of claim 1, wherein the final color prediction includes values for each of a plurality of wavelengths.

8. The method of claim 1, wherein the output from the trained machine learning model is a second color prediction.

9. The method of claim 1, wherein the analytical model is a model based on Kubelka-Munk (K-M) theory.

10. The method of claim 1, wherein the trained machine learning model is a plurality of models, wherein the plurality of models generates respective predictions, and wherein the output is based on the respective predictions.

11. A coating prepared using the method of claim 1.

12. A method for predicting a color for a coating, comprising:
training a machine learning model based on a training data set, the training data set includes at least measured values for a plurality of formulations;
receiving an input formulation;
generating a first color prediction using an analytical model based on the input formulation;
generating an adjustment to the first color prediction using the trained machine learning model; and
determining a color prediction for the input formulation based at least in part on the first color prediction or the adjustment.

13. The method of claim 12, wherein the training data set further includes color predictions for the plurality of formulations obtained using the analytical model.

14. The method of claim 12, wherein the measured values are spectral reflectance values.

15. The method of claim 12, wherein the first color prediction includes one or more spectral reflectance values, wherein the one or more spectral reflectance values correspond to respective wavelengths.

16. The method of claim 15, wherein the adjustment includes one or more residual values respectively associated with the one or more spectral reflectance values of the first color prediction.

17. The method of claim 16, wherein the color prediction determined is based on a summation of the one or more residual values and respective spectral reflectance values of the first color prediction.

18. The method of claim 15, wherein the adjustment is a second color prediction specifying one or more second spectral reflectance values, the second spectral reflectance values representing a correction to the one or more spectral reflectance values of the first color prediction.

19. The method of claim 12, wherein training the machine learning model further comprises training a plurality of machine learning models, wherein each model of the plurality machine learning models generates an output corresponding to a particular wavelength.

20. The method of claim 19, wherein the adjustment to the first color prediction is based on a combination of respective outputs of the plurality of machine learning models.
